(19) United States
(12) Patent Application Publication
(10) Pub. No.: US 2005/0010056 A1
(43) Pub. Date: Jan. 13, 2005

(54) Concentrating aqueous fractions of lisinopril by reverse osmosis

(76) Inventors: Janez Rzen, Ljubljana (SK); David Senica, Ljubljana (SK); Pavel Drnovsek, Domzale (SK)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/496,780

(22) PCT Filed: Nov. 8, 2002
(86) PCT No.: PCT/SI02/00025

(30) Foreign Application Priority Data
Nov. 26, 2001 (SI) P-200100300

(51) Int. Cl. C 07D 2 7/12

(52) U.S. Cl. 548/537

(57) ABSTRACT

The present invention describes a process for purification and isolation of lisinopril where the reverse osmosis is used for concentrating the aqueous fractions.

### TECHNICAL FIELD OF THE INVENTION

(IPC: C 07 K 5/06)

The invention belongs to the field of chemical engineering and deals with concentrating by means of the reverse osmosis (RO).

In a narrower sense, the invention deals with a process of concentrating aqueous fractions of lisinopril by means of the reverse osmosis.

### TECHNICAL PROBLEM

After the completed synthesis of lisinopril there are dissolved in water, in addition to the desired product, also numerous undesired impurities such as lisinopril isomers, the remains of the starting raw materials and their derivatives, by-products of reactions and similar. Most undesired impurities are removed by chromatographic purification.

In the process of purification and isolation of lisinopril it is necessary (sometimes even several times) to concentrate the aqueous fractions, wherefor a one-step vacuum circular evaporator has been used so far. The aqueous fractions were concentrated to a desired concentration of lisinopril by evaporation of water at an absolute pressure lower than 100 mbars. For the evaporation of large quantities of water, very productive evaporators are required and the operation costs are relatively high.

Our invention arises from the need for a more efficient technological solution for concentrating the aqueous fractions of lisinopril, which would be more efficient and more economical than vacuum evaporation, both prior to the final crystallization and in other steps of the synthesis process.

### PRIOR ART

Lisinopril is a pharmaceutically active compound from the group of angiotensin-converting enzyme (ACE) inhibitors used for the treatment of hypertension. Lisinopril dihydrate, chemically N-[N-[(1S)-carboxy-3-phenylpropyl]-L-lisyl]-L-proline dihydrate is prepared by a multi-step synthesis which is described e.g. in the article J. Org. Chem. 53 (1988) 836, in U.S. Pat. No. 4,374,829, wherein the purification is carried out by adsorption chromatography or by lyophilization, or by some other process.

In some cases the reverse osmosis can be used for concentrating the solutions instead of classical techniques. The investment, operation and maintenance costs are lower, the reverse osmosis can operate at lower temperatures, it is simple to automate it as well as to scale it up from laboratory or pilot scales to the industrial scale. The usefulness of the reverse osmosis is limited by the chemical and physical resistance of the membranes. Depending on their materials and construction, the membranes can only be used in specific pressure, temperature and pH ranges. Too high pressures and temperatures and too low or too high pH's can irreversibly damage the membranes. The membranes can also be irreversibly damaged by some organic solvents and inorganic compounds. The reverse osmosis is not useful for concentrating solutions of solutes with a low solubility either. By a proper selection of the membranes and operating conditions, however, the reverse osmosis can be efficiently used for various purposes.

In the patent and other literature from this field, no literature source describing the use of the reverse osmosis method in a process of purification and/or isolation of lisinopril has been found.

### DESCRIPTION OF NOVEL SOLUTION WITH EXAMPLES

The object of the invention is a novel process for the purification and isolation of lisinopril synthesized in a multi-step synthesis. After the completed synthesis, lisinopril is chromatographically purified, the chromatographic fractions are concentrated and lisinopril is isolated by precipitation. The chromatographic fractions are concentrated to the desired concentration by the use of the reverse osmosis, which is more economical than the evaporation of large quantities of water in a simple one-stage vacuum circular evaporator. The usefulness of the reverse osmosis for concentrating was examined at various operating conditions on various scales.

After column separation, the concentrating of the aqueous solution of lisinopril by the reverse osmosis was performed on a pilot scale in a batch mode and in a continuous mode. A membrane element of the spiral-wound type with a filter surface area of 7.6 m² was used.

The operating conditions for concentrating the aqueous fractions of lisinopril were determined in the batch mode of operation. From the membrane module the concentrate flowed back into the feed tank and the permeate was removed or recycled back to column separation. The concentration range of lisinopril was from 1 g/L to 100 g/L.

The processes, the continuous and/or the batch ones, can run in a temperature range from some degrees above freezing point up to about 45° C., preferably at about 5-30° C. At a prior acidification of the solution, the operating pH can be in the range between about 4 and about 11, otherwise between about 7 and about 11, preferably about 9-10.5. The operating pressures can be between about 5 and about 60 bars, preferably between about 5 and about 41 bars, most preferably about 10-35 bars. The permeate recovery (i.e. an intrinsic property of the membrane), which is the ratio between the permeate flow rate and the sum of the permeate and the concentrate flow rates (i.e. flow rate of the feed flow) expressed in %, can be between about 5% and about 30%, preferably at about 10-25%. Depending on the operating conditions, the fluxes of the permeate were from about 5 to about 45 L/hm², preferably about 10-35 L/hm².

As an alternative to the membrane modules for the reverse osmosis and instead of them, there can be used nano-filtering membrane modules of various constructions such as e.g. plate and frame or spiral-wound types or the like and made of various materials (organic or inorganic).

The concentrated solution of lisinopril is crystallized according to known processes. The product is raw lisinopril, which is then converted into the final form, namely lisinopril dihydrate.

The invention is explained but in no way limited by the following Examples.

### EXAMPLE 1

The chromatographic fractions, which after column separation contained 1% of acetonitrile, 0.5% of ammonia and 1 g/L of lisinopril, were concentrated in the batch mode in a pilot plant for the reverse osmosis. From the feed tank, the solution was pumped by high-pressure pumps into the reverse osmosis membrane module. At the beginning of concentrating, the drop of the pressure on the membrane amounted to about 10 bars. During the concentrating the flow rates of the permeate and the concentrate were maintained approximately constant—the flow rate of the permeate at about 140 L/h and the flow rate of the concentrate at about 640 L/h. After the completed concentrating, the concentration of lisinopril in the concentrate was 100 g/L and the drop of the pressure on the membranes was about 27 bars.

### EXAMPLE 2

The chromatographic fractions, which after column separation contained 1% of acetonitrile, 0.5% of ammonia in 1 g/L of lisinopril, were concentrated in the batch mode in a pilot plant for the reverse osmosis. From the feed tank, the solution was pumped by high-pressure pumps into the reverse osmosis membrane module. At the beginning of concentrating, the drop of the pressure on the membrane amounted to about 20 bars. During the concentrating the flow rates of the permeate and the concentrate were maintained approximately constant—the flow rate of the permeate at about 220 L/h and the flow rate of the concentrate at about 950 L/h. After the completed concentrating, the concentration of lisinopril in the concentrate was 100 g/L and the drop of the pressure on the membranes was about 37 bars.

1. A process for the purification and obtaining of lisinopril, characterized in that, in one or more steps, it comprises concentrating an aqueous solution of lisinopril by means of the reverse osmosis.

2. A process for the purification of lisinopril according to claim 1, characterized in that an aqueous solution of lisinopril is concentrated by the reverse osmosis prior to crystallization.

3. A process for the purification of lisinopril according to claims 1, characterized in that the reverse osmosis is performed at operating temperatures up to about 45° C.

4. A process for the purification of lisinopril according to claim 3, characterized in that the reverse osmosis is performed at operating temperatures between about 5° C. and about 30° C.

5. A process for the purification of lisinopril according to claims 1, characterized in that the reverse osmosis is performed at a pH between about 4 and about 11.

6. A process for the purification of lisinopril according to claim 5, characterized in that the reverse osmosis is performed at a pH between about 9 and about 10.5.

7. A process for the purification of lisinopril according to claims 1, characterized in that the reverse osmosis is performed at pressures between about 5 and about 60 bars.

8. A process for the purification of lisinopril according to claim 7, characterized in that the reverse osmosis is performed at pressures between about 5 and about 41 bars.

9. A process for the purification of lisinopril according to claim 8, characterized in that the reverse osmosis is performed at pressures between about 10 and about 35 bars.

10. A process for the purification of lisinopril according to claims 1, characterized in that the reverse osmosis is performed at a permeate recovery between about 5% and about 30%.

11. A process for the purification of lisinopril according to claim 10, characterized in that the reverse osmosis is performed at a permeate recovery between about 10% and about 25%.
